(19) United States
(12) Patent Application Publication
(10) Pub. No.: US 2010/0251435 A1
(43) Pub. Date: Sep. 30, 2010

(54) METHOD FOR GROWING SUGARCANE

(75) Inventors: Paulo Aramaki, Sao Paulo (BR); Jakob Andreas Leunberger, Basel (CH); Antonio Carlos Nascimento, San Paulo (BR); Jose Geraldo Martins Dos Santos, San Paulo (BR)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 12/665,444

(22) PCT Filed: Jun. 9, 2008
(86) PCT No.: PCT/EP08/04573

§ 371 Date: May 24, 2010

(30) Foreign Application Priority Data
Jun. 22, 2007 (EP) 07012294.0

(51) Int. Cl. A01H 5/00; A01G 1/00; A01C 11/00

(52) U.S. Cl. 800/320; 47/58.1 R; 111/200

(57) ABSTRACT

The present invention relates to a method of growing a gramineous crop plant comprising the steps of providing a stem section of a gramineous crop plant which section comprises at least one node, planting said section, and growing a gramineous crop plant from said planted stem section. It also relates to a stem section of a gramineous crop plant comprising at least one node.

The present invention relates to a method of growing a gramineous crop plant comprising the steps of providing a stem section of a gramineous crop plant which section comprises at least one node, planting said section, and growing a gramineous crop plant from said planted stem section. It also relates to a stem section of a gramineous crop plant comprising at least one node.

Sugar cane is a commercially important gramineous plant. Sugar cane acreage is increasing, and its uses include the production of sugar, Falernum, molasses, rum, cachaça (the national spirit of Brazil) and ethanol for fuel. The bagasse that remains after sugar cane crushing can be used to provide both heat energy, used in the mill, and electricity, which is typically sold to the consumer electricity grid and as a feedstock for the production of ethanol. Therefore better agricultural practices for sugar cane growth are sought.

A seed of sugar cane is a dry one-seeded fruit or caryopsis formed from a single carpel, the ovary wall (pericarp) being united with the seed-coat (testa). The seeds are ovate, yellowish brown and very small, about 1 mm long. However, for commercial agriculture, the seed of a sugar cane is not sown or planted, but the cane cuttings (also known as a stem section (or part of a stalk or culm or seedling)) of 40-50 cm in length are placed horizontally in furrows which are generally wide at ground level & deep (40 to 50 cm wide and 30 to 40 cm deep), and then lightly covered with soil (see FIG. 1).

The stem of sugar cane comprises generally several nodes and internodes as in other grasses. The term “node” means the part of the stem of a plant from which a leaf, branch, or aerial root grows; each plant has many nodes. At the position of each node, a bud (or gemma) forms, that can grow to yield the crop. Suitable material for cuttings are pieces of cane cut from 8-14 month old healthy plants, with the older basal buds or buds in the middle to top of the stem germinating stronger and faster. The cuttings are taken from plants which themselves have generally grown from cuttings.

The nodes range from 10 to 25 cm apart along the above-ground section of the stem. At each node a broad leaf rises which consists of a sheaf or base and the leaf blade. The sheaf is attached to the stem at the node and at that point entirely surrounds the stem with edges overlapping. The sheath from one node encircles the stem up to the next node above and may overlap the base of the leaf on the next higher node. The leaf blade is very long and narrow, varying in width from 2.5 to 7.5 cm and up to 1.5 m or more in length. Also, at each node along the stem is a bud, protected under the leaf sheath. When stem sections are planted by laying them horizontally and covering with soil a new stem grows from the bud, and roots grow from the base of the new stem. The stem branches below ground so several may rise as a clump from the growth of the bud at a node.

In planting sugar cane fields, mature cane stems are cut into sections, either manually in the furrows or by automation and laid horizontally in furrows. In continental United States cuttings with several nodes are laid while in tropical countries sections with only 2 or 3 nodes are commonly used—since temperatures for growth are more favourable.

The cuttings can be prepared either manually or by mechanical means. Manual preparation involves manually cutting the longer cuttings in the furrow into smaller stem sections having on average three buds, and so a stem section could unintentionally have one bud because of the overlap between the cuttings in the furrow. When mechanical means are used for preparing the cuttings, the stem sections generally have 2 to 3 buds per stem section and these are then placed in the furrows also with aid of mechanical means. Once planted, a stand of cane can be harvested several times; after each harvest, the cane sends up new stems, called ratoons. Usually, each successive harvest gives a smaller yield, and eventually the declining yields justify replanting. Depending on agricultural practice, two to ten harvests may be possible between plantings. After planting, the crop is sprayed with water, fertilizer, and pesticides, such as herbicides and insecticides.

Planting is in rows about 1.8 m apart to make possible cultivation and use of herbicides for early weed control. As plants become tall lower leaves along the stems are sbaded and die. These ultimately drop off, so only leaves toward the top remain green and active.

These existing agricultural practices with for example, sugar cane, show several disadvantages such as the requirement of workmanship to cut the stem, use of different kinds of bulky machines, many steps and low efficiency. This scenario usually leads to high costs of operation and logistic and undesirable risks for people working in field when cutting the stems. Additionally, one of the greatest disadvantages is that the cutting is cut in a long length of about 40 cm, especially when automated, more specifically about 37 cm, in order to ensure that there will be at least two or three buds (or also known as gemmas) per part of cutting, which requires large areas for processing and incurs higher costs. Further, once cut, the larger stem sections require big areas to stock, bringing further increased costs for the process. Also, the planting of the known stem sections requires a high weight of stem sections per hectare, such as 16-18 ton/ha (by mechanic planting) or 12-16 ton/ha (by conventional planting).

Applicant has found that using a certain defined stem section and planting the stem sections (or cuttings) in a field so that a substantial proportion of the stem sections of the crop that is planted or sown has one bud per stem section (see FIG. 2), many of the disadvantages of the state of art can be overcome because single bud carretels (stem sections) are much smaller and lighter than conventional 3-bud stem sections. However, single bud carretels are more susceptible to pests, disease, and dehydration, and therefore the rate of emergence from single bud carretels is poor. Therefore there exists a need to improve the rate of emergence from single bud carretels.

According to the present invention, there is provided a method of growing a gramineous crop plant comprising the steps of providing a stem section of a gramineous crop plant which section comprises at least one node, planting said section, and growing a gramineous crop plant from said planted stem section, wherein the stem section is stored at a temperature below 15° C. before it is planted.

According to the present invention, there is provided a method of propagating a gramineous crop plant comprising the steps of: providing more than one stem section from a gramineous crop plant by cutting the stem of said plant, wherein each section comprises at least one node; planting said multiple sections; and growing gramineous crop plants from said planted stem sections; wherein the stem sections are stored at a temperature below 15° C. before it is planted. Growing and propagation are related—before propagation is possible, the stem sections must be converted into viable plants by a process of growth and development, hereinafter referred to as “growth”.

According to the present invention, there is provided a stem section of a gramineous crop plant, characterized in that it comprises at least one node, and has been stored at a temperature below 15° C. before it is planted. Suitably the stem section is stored at a temperature from 1 to 15° C. before it is planted.

Suitably, the stem section is stored at a temperature below 10° C. before it is planted. Suitably, the stem section is stored at a temperature from 1 to 10° C. before it is planted. More suitably, the stem section is stored at a temperature from about 6 to about 8° C. before it is planted.

According to the present invention, there is provided a method of growing a gramineous crop plant comprising the steps of: preparing a stem section of a gramineous crop plant by cutting the stem of said plant, wherein the section comprises only one node; planting said section; and growing a gramineous crop plant from said planted stem section; wherein the stem section is planted within about 2 months after it has been prepared.

According to the present invention, there is provided a method of propagating a gramineous crop plant comprising the steps of: preparing more than one stem section from a gramineous crop plant by cutting the stem of said plant, wherein each section comprises only one node; planting said multiple sections; and growing gramineous crop plants from said planted stem sections; wherein the stem sections are planted within about 2 months after they have been prepared.

According to the present invention, there is provided a stem section of a gramineous crop plant that has been prepared by cutting the stem of said plant, characterized in that it comprises only one node, and is planted within about 2 months after it has been prepared.

Suitably, the stem section is planted within about 1 month after it has been prepared. More suitably, the stem section is planted within about 2 weeks after it has been prepared.

In one embodiment of the present invention, the stem section comprises only one node.

Suitably the stem section is from about 2 to about 12 cm in length. More suitably, it is from about 3 to about 8 cm in length, especially from 3.5 to 4.5 cm in length.

The stem section may planted in any suitable orientation. In one embodiment, the stem section is planted in an essentially horizontal position in a furrow.

In one aspect of the invention, the cut ends of the stem section is covered with a coating or binder such as paraffin, to reduce water loss and further enhance shelf life.

The present invention may be applied to any gramineous crop plant. Gramineous crop plants are from the genus Graminae, which is an alternative family name for Poaceae. Suitably the gramineous crop plant is from the sub-tribe Saccharinae. Suitably, the gramineous crop plant is selected from the group consisting of Saccharum spp., Sorghum spp., and bamboo. More suitably, it is Saccharum spp. (sugar cane).

Bamboo means any of various usually woody, temperate or tropical grasses of the genera Arundinaria, Bambusa, Dendrocalamus, Phylloslachys, or Sasa.

According to the present invention, there is provided the use of a sugar cane stem section as defined above in growing a sugar cane crop.

The present invention, therefore, allows cost-effective methods having logistic advantages for cultivating a crop, such as sugar cane, through improved handling, storage, planting and growth. Further, the gramineous crop plant and part of the plant that grows at a later point in time is able to more effectively withstand pest and/or pathogen pressure.

### DESCRIPTION OF THE FIGURES

The present invention will be described in more details based on the following figures:

FIG. 1 illustrates a conventional method for cultivating sugarcane, in which stem sections have 3 or more buds are planted in a furrow.

FIG. 2 illustrates an embodiment of the present invention, in which stem sections having only one bud are planted in a furrow.

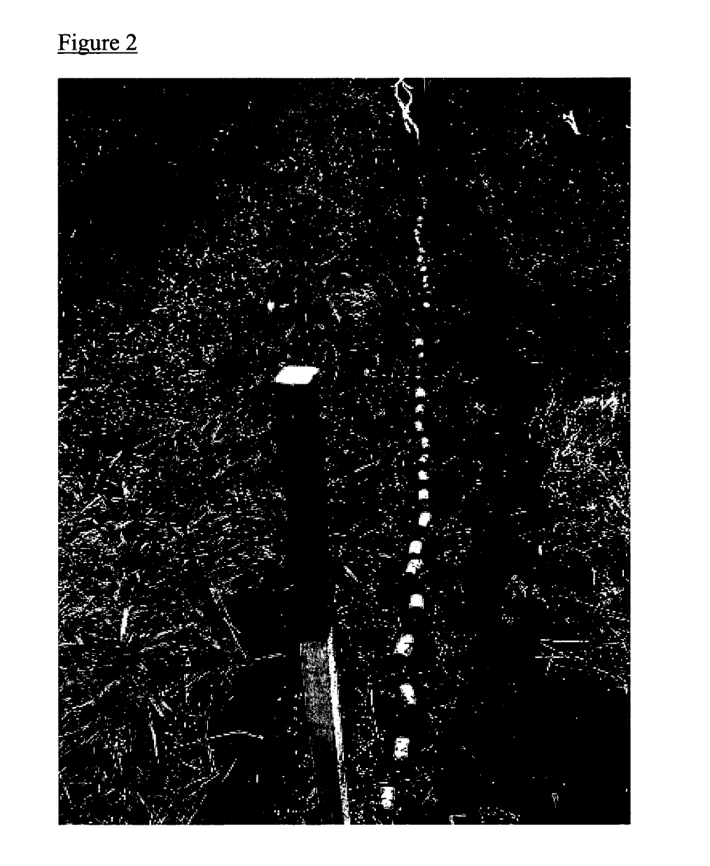

The invention is described in detail below.

Examples of crops suitable for the present invention include sugar cane, bamboo and sorghum. These crop plants are generally planted or sown in long stem sections in a furrow horizontally.

Sugarcane or sugar cane (Saccharum) is a genus of 6 to 37 species (depending on taxonomic interpretation) of tall grasses (family Poaceae, tribe Andropogoneae), native to warm temperate to tropical regions of the Old World. They have stout, jointed, fibrous stems that are rich in sugar and measure 2 to 6 meters tall. All of the sugarcane species interbreed, and the major commercial cultivars are complex hybrids.

Specific examples of species include Saccharum arundinaceum, Saccharum bengalense, Saccharum edule, Saccharum officinarum, Saccharum procerum, Saccharum ravennae, Saccharum robustum, Saccharum sinense, Saccharum spontaneum

In respect of sugar cane, there are several varieties or cultivars and germplasms which can be used in combination with the present invention for improved methods of growing a sugar cane crop.

The gramineous crop plant may be transgenic or non-transgenic. Transgenic gramineous crop plants are produced by transformation via recombinant DNA technology in such a way that they are—for instance—capable of synthesizing selectively acting toxins as are known, for example, from toxin-producing invertebrates, especially of the phylum Arthropoda, as can be obtained from Bacillus thuringiensis strains; or as are known from plants, such as lectins; or in the alternative capable of expressing a herbicidal or fungicidal resistance. Examples of such toxins, or transgenic plants which are capable of synthesizing such toxins are known to the skilled man. Also suitable are crop plants with particular trait characteristics built in, such as drought resistance or improved quality, such as enhanced sugar or ethanol content.

A plant variety exhibiting a trait of interest can be obtained by introducing into the plant a nucleic acid sequence associated with a trait of interest. Methods for preparing nucleic acid sequences, combining them with control sequences such as promoters and transcriptional or translational termination regions, and introducing said sequences into plants so that they express said sequences are well known in the art.

The genetic properties engineered into transgenic seeds and plants are passed on by sexual reproduction or vegetative growth and can thus be maintained and propagated in progeny plants. Generally, maintenance and propagation make use of known agricultural methods developed to fit specific purposes such as tilling, sowing or harvesting.

Examples of common sugar cane cultivars are RB 72-454; RB 85-5156; RB 85-5453; RB 83-5486; RB 85-5536; RB 86-7515; RB 84-5257; RB 85-5113; RB 85-5035; RB 84-5210; RB 92-8064; SP-72-1011; SP 79-1011; SP 91-3011; SP 77-5181; SP 84-1431; SP 83-5073; SP 85-3877; SP 83-2847; SP 84-5560; SP 81-3250; SP 80-3280; SP 80-1816; SP 87-396; SP 80-1842; SP 86-42; SP 91-1049; SP 90-3414; SP 90-1638; SP 86-155; SP 87-365; SP 84-2025; SP 89-1115; I.A.C.91-2195; I.A.C.96-2210; I.A.C 87-3396; I.A.C.93-6006; I.A.C.91-2218; and I.A.C.91-5155. A preferred cultivar of the sugarcane is known as SP-72-1011.

The stem section (or ‘cutting’, or part of the stalk or culm) for planting according to the invention preferably has defined characteristics, such as having at least one bud (or gemma), having only one bud and/or having a defined length. Suitably the stem section has only one bud.

In the context of the present invention, the term ‘bud’ also encompasses the node at which a bud is capable of forming, since the bud itself may not have formed at the time of cutting or planting.

In one embodiment, the stem section is from about 2 to about 20 cm in length, suitably from about 2 to about 12 cm, more suitably from about 3 to about 8 cm, more suitably from about 3.5 to 4.5 cm, and especially about 4 cm in length.

In a further embodiment, the stem section is of a minimum length that the section contains at least one bud. In a further embodiment, the stem section is of a maximum length that is only contains one bud. Suitably, the stem section is from about 3 to about 8 cm in length, and comprises only one bud.

The present invention is suitable for the different types of nodes (e.g. tall root band, contricted root band, conoidal root band and obconoidal root band) and internodes (e.g. cylindrical, tumescent, bobbin-shaped, conoidal, obconoidal and concave-convex).

A stem section containing one bud can be obtained by manually cutting the cutting to the desired lengths (for example, with a machete) or by mechanical or automated means. Suitably, the stem section is cut by a device that identifies the position of a bud or node in a stem, and automatically cuts the stem section either side of the bud or node. Alternatively, the stem section is prepared by cutting a stem at random and using a device to automatically select the resulting stem sections that possess at least one bud. Either device may use sensors, such as visual or electromagnetic sensors, to determine the presence of absence of a bud or node.

Suitably, the cutting process does not require any human intervention, and can be performed at a high throughput, for example cutting up to 100, 500 or even 1000 stem sections per minute.

Suitably, the stem section is prepared by making two cross-sectional cuts through the stem, one above and one below the position of the node at which the bud forms.

Also envisaged in the present invention is cutting of the long stem sections to a predetermined length by any suitable cutting means, such as a circular saw, laser cutting system, plasma cutting system, high pressure water cutting system, stamping device, shear device, or suitable blades such as a knife or scythe. The cutting means may also have an image analysis and adequate controls to allow precise cutting of the stem section to a predetermined length and/or having only one bud. The means for cutting should not unacceptably damage the germination potential of the bud.

An example of a mechanical means is described in JP 10-313611 in which sugarcane is cut with the disk cutter of a rotating type, in order to obtain the desired length of the stem section.

In a preferred embodiment of the invention, the stem sections according to the invention are obtained by a mechanical or non-manual means from longer lengths of stem sections.

The stem sections can be planted or sown manually or with a mechanical means. In one embodiment, the stem sections are applied to the soil so that there are from about 2 to about 200, preferably about 2 to about 100, more preferably about 4 to about 75, especially about 5 to about 40, advantageously 6 to 30, buds per linear meter in the soil.

In an embodiment, the distance between each stem section applied to soil varies from 0.5 to 50 cm, suitably from 1 to 40 cm, more suitably from 2 to 30 cm, especially from 3 to 20 cm.

The planting or sowing according to the invention can follow conventional practices, such as planting or sowing in a furrow, whether by hand or by a mechanical means; alternatively each stem section can be planted or sown in discrete positions in a field, whether by hand or by a mechanical means.

In the instance of in-furrow planting or sowing, a particular advantage of the invention is that the width of the furrow is substantially less than conventional planting of sugar cane. Typically the width at ground level can be 10-20 cm and the depth is 30-40 cm: this minimizes ploughing and enables shallow harrowing.

In an embodiment of the invention, the stem sections may also be planted with a device such as a modified potato tuber planting machine with belt and buckets. Suitably, each stem section is of uniform shape and size to facilitate automatic planting. Methods for achieving stem sections of a uniform shape and size can be via known technologies, such as pelleting, encapsulation and coating.

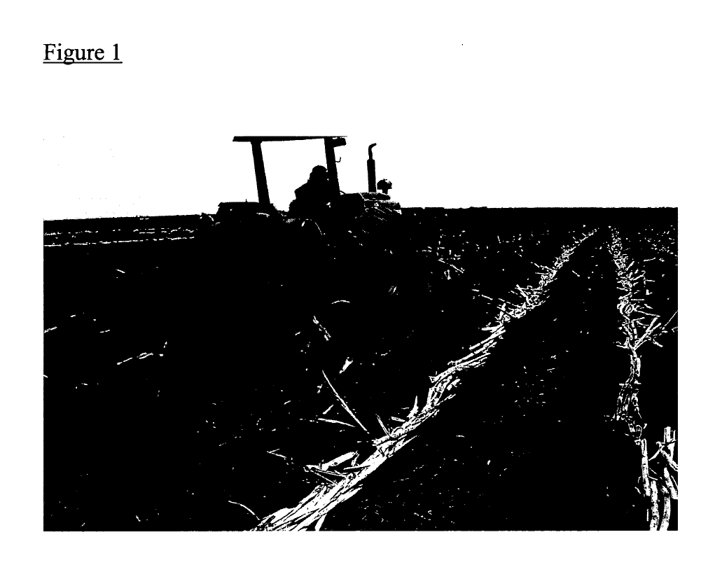

Contrary to the conventional art, as illustrated in FIG. 1, where 12 to 20 ton of sugar cane cuttings are required to plant 1 hectare, the present invention only requires about 0.5 to 5 ton sugar cane cuttings per hectare.

The aspect of preparing the field, opening the furrow and closing the furrow, drilling the holes or planting the stem sections according to the invention can be carried out by conventional methods. However, the present invention enables use of less bulky machinery for cultivating sugar cane.

After the crop plant is grown, conventional methods of harvesting can be employed.

A particular advantage of the present invention is that successive planting of stem sections requires minimum tillage or harrowing of the field since the defined section can be planted between the existing rows of sugar cane crops since the furrows are not as wide as conventional practice. Therefore, in one embodiment of the present invention, the crop defined in the first aspect, preferably sugarcane, can be cultivated through a farming practice referred to as “no till planting or minimum tillage”, which is an innovation in the method for cultivating this kind of crop. Soya, corn and wheat, for example, are known to be cultivated by no till planting; however, sugar cane, for example, is not cultivated by this practice at present.

Conventional sugar cane preparatory practices such as warm water treatment of the cuttings, treatment of the cuttings with mercury preparations, not planting the cuttings too deep, managing the direct contact of the cuttings with fertilizer, ensuring sufficient soil moisture, application of pesticides (e.g. herbicide, insecticide, nematicide, etc) to the field and plant, and ensuring good soil aeration and soil temperature, can also be carried out prior to, during or after the planting of the defined stem sections of the invention.

The present invention provides the following advantages regarding the use of a predetermined length of the stem section:
Requirement of a smaller area to prepare and stock and the defined stem sections (or culms, or seedlings). For example, to plant about 1.5 Mha of sugar cane cultivated area conventional and mechanized cultivation methods usually require about 120K ha of land and 250K ha of land respectively, and about 12 ton sugar cane cuttings per hectare and 18 ton sugar cane cuttings per hectare respectively; in contrast, using the present invention, only 30 to 80K ha of land and about 1.5 ton stem sections per hectare are required to produce the mentioned about 1.5 Mha of cultivated area. Therefore, a much smaller area of nursery land is required to generate sufficient cuttings to plant a given cultivation area. Further, the weight of cuttings required to plant the cultivation area is reduced by approximately 10 fold.
Reduced exposure to pesticides for workers in the field;
Minimizes soil erosion, especially in tropical climates with heavy rainfall due to the use of less bulky machinery, and the ability to use minimum tillage cultivation;
Minimizes soil compaction because of less bulky machinery needed to transport and plant cuttings;
Preserves soil moisture because of ability to employ minimum tillage or no till cultivation methods;
Reduced operational costs due to reduced manual labour and the ability to use smaller machinery;
Easier cultivating method; and
Ability to plant in a broadened planting window, since the use of smaller and lighter machinery in fields may be possible even when the ground is wet.

In one aspect of the present invention, the application of conventional pesticides such as insecticides, fungicides, nematicides, miticides, termicicides, acaricides and molluscicides is useful to control pests and diseases that affect the emergence of buds into new plants, and herbicides is useful to provide weed control at the growing site to minimise the presence of unwanted plants that compete for light, nutrients and water. Important pests of sugarcane include: the larvae of some lepidoptera species such as the turnip moth, sugarcane borer (Diatraea saccharalis), early shoot borer (Chilo infescatellus), internode borer (Chilo saccharifagus indicus), top borer (Scirpophaga excerptalis) and Mexican rice borer (Eoreuma loftini); leaf-cutting ants; termites (such as Coptotermes heimi, Microtermes obesi, Odontotermes assmuthi); spittlebugs (especially Mahanarva fimbriolata and Deois flavopicta); scale insects (such as Melanaspis glomerata); pyrilla (such as Pyrilla purpusilla); beetles (such as Migdolus fryanus); and nematodes (such as Pratylenchus spp., Meloidogyne spp., Helicotylenchus spp., Rotylenchus spp., and Scutellonema spp).

Sugar cane is also affected by disease, such as:

Bacterial diseases

| | |
|---|---|
| Gumming disease | Xanthomonas campestris pv. |
| | vasculorum |
| Leaf scald | Xanthomonas albilineans |
| Mottled stripe | Herbaspirillum rubrisubalbicans |
| Ratoon stunting disease | Leifsonia xyli subsp. xyli |
| Red stripe (top rot) | Acidovorax avenae |

Fungal diseases

| | |
|---|---|
| Banded sclerotial (leaf) disease | Thanatephorus cucumeris = |
| | Pellicularia sasakii |
| | Rhizoctonia solani [anamorph] |
| Black rot | Ceratocystis adiposa |
| | Chalara sp. [anamorph] |
| Black stripe | Cercospora atrofiliformis |
| Brown spot | Cercospora longipes |
| Brown stripe | Cochliobolus stenospilus |
| | Bipolaris stenospila [anamorph] |
| Downy mildew | Peronosclerospora sacchari = |
| | Sclerospora sacchari |
| Downy mildew, leaf splitting | Peronosclerospora miscanthi = |
| form | Sclerospora miscanthi |
| | Mycosphaerella striatiformans |
| Eye spot | Bipolaris sacchari = |
| | Helminthosporium sacchari |
| Fusarium sett and stem rot | Gibberella fujikuroi |
| | Fusarium moniliforme [anamorph] |
| | G. subglutinans |
| Iliau | Clypeoporthe iliau = |
| | Gnomonia iliau |
| | Phaeocytostroma iliau [anamorph] |
| Leaf blast | Didymosphaeria taiwanensis |
| Leaf blight | Leptosphaeria taiwanensis |
| | Stagonospora taiwanensis [anamorph] |
| Leaf scorch | Stagonospora sacchari |
| Marasmius sheath and shoot | Marasmiellus stenophyllus = |
| blight | Marasmius stenophyllus |
| Myriogenospora leaf binding | Myriogenospora aciculispora |
| (tangle top) | |
| Phyllosticta leaf spot | Phyllosticta hawaiiensis |
| Phytophthora rot of cuttings | Phytophthora spp. |
| | P. megasperma |
| Pineapple disease | Ceratocystis paradoxa |
| | Chalara paradoxa = |
| | Thielaviopsis paradoxa |
| | [anamorph] |
| Pokkah boeng (that may have | Gibberella fujikuroi |
| knife cut symptoms) | Fusarium moniliforme [anamorph] |
| | G. subglutinans |
| Red leaf spot (purple spot) | Dimeriella sacchari |
| Red rot | Glomerella tucumanensis = |
| | Physalospora tucumanesis |
| | Colletotrichum falcatum [anamorph] |
| Red rot of leaf sheath and sprout | Athelia rolsfii = |
| rot | Pellicularia rolfsii |
| | Sclerotium rolfsii [anamorph] |
| Red spot of leaf sheath | Mycovellosiella vaginae = |
| | Cercospora vaginae |
| Rhizoctonia sheath and shoot rot | Rhizoctonia solani |
| Rind disease (sour rot) | Phaeocytostroma sacchari = |
| | Pleocyta sacchari = |
| | Melanconium sacchariv |
| Ring spot | Leptosphaeria sacchari |
| | Phyllosticta sp. [anamorph] |
| Root rots | Marasmius sacchari |
| | Pythium arrhenomanes |
| | P. graminicola |
| | Rhizoctonia sp. |
| | Unidentified Oomycete |
| Rust, common | Puccinia melanocephala - P. erianthi |
| Rust, orange | Puccinia kuehnii |
| Schizophyllum rot | Schizophyllum commune |
| Sclerophthora disease | Sclerophthora macrospora |
| Seedling blight | Alternaria alternata |
| | Bipolaris sacchari |
| | Cochliobolus hawaiiensis |
| | Bipolaris hawaiiensis [anamorph] |
| | C. lunatus |
| | Curvularia lunata [anamorph] |
| | Curvularia senegalensis |
| | Setosphaeria rostrata |
| | Exserohilum rostratum [anamorph] = |
| | Drechslera halodes |
| Sheath rot | Cytospora sacchari |
| Smut, culmicolous | Ustilago scitaminea |
| Target blotch | Helminthosporium sp. |
| Veneer blotch | Deightoniella papuana |
| White rash | Elsinoe sacchari |
| | Sphaceloma sacchari [anamorph] |
| Wilt | Fusarium sacchari = |
| | Cephalosporium sacchari |
| Yellow spot | Mycovellosiella koepkei = |
| | Cercospora koepkei |
| Zonate leaf spot | Gloeocercospora sorghi |

Viral diseases

| | |
|---|---|
| Chlorotic streak | Virus (putative) |
| Dwarf | Sugarcane dwarf virus |
| Fiji disease | Sugarcane Fiji disease virus |
| Grassy shoot | Phytoplasma |
| Mosaic | Sugarcane mosaic virus |
| Sereh | Virus (putative) |
| Streak disease | Maize streak virus, sugarcane strain |
| White leaf | Phytoplasma |

Compounds that exhibit either stimulatory or growth-promoting activity can be applied to the soil or to the stem section itself. Such compounds include sugars, nutrients, fertilizers, micronutrient donors, biological agents, inoculants (such as nitrogen fixing bacteria), antibiotics and the like. Fertilisers may be sources of nitrogen, phosphorus or potassium, or mixtures of two of more of these. Certain crop protection chemicals, such as the insecticide thiamethoxam, can also have a stimulatory or growth-promoting activity.

Compounds that exhibit inhibitory activity towards pests and/or pathogens can be applied to the soil or to the stem section itself. Such compounds include plant protection chemicals, such as pesticides including insecticides, fungicides and nematicides. Suitable insecticides include thiamethoxam (e.g. Cruiser®), imidacloprid, clothianidin, abamectin (e.g. AVICTA®), fipronil (e.g. Regent®), carbofuran (e.g. Furadan®), and chlorantraniprole. Some of these insecticides also have activity against termites, nematodes and/or molluscs. Suitable fungicides include azoxystrobin, mefenoxam, cyproconazole, fludioxonil, triadimenol (e.g. Bayfidan®). Mixtures of more than one pesticide may be applied to the soil or stem section, for example a mixture of thiamethoxam and abamectin, a mixture of azoxystrobin, mefenoxam and fludioxonil (e.g. Dynasty®), and a mixture of azoxystrobin and cyproconazole (Priori Xtra®), or a mixture of fipronil, carbofuran and triadimenol. Suitable nematicides include carbamates such as carbofuran, carbosulfan, thiodicarb, aldicarb (e.g. Temik®), furathiocarb, cadusafos (e.g. Rugby®) and Vidat®.

Compounds can be applied to the field before, during and/or after the planting or sowing of the sugar cane cuttings to control pathogenic and/or pest damage and to promote the growth of the crop.

Further, weed control agents can also be applied to ensure control of undesired vegetation. In one embodiment, one or more compounds that exhibit safening activity against pesticide (e.g. a safener) can also be applied to the soil or locus of the stem section, in particular when to manage the phytotoxicity of a herbicide.

The pesticides, including weed control agents, and mixtures thereof can be of any type and formulation suitable for the circumstances and are known to a skilled person.

Methods for applying the compounds to the soil or locus of the stem section are known to a skilled person, and conventionally practiced.

In an embodiment, one or more neonicotinoid compounds, strobilurin compounds and/or fipronil are applied to the stem sections, or to the locus of the stem sections in the soil.

Examples of neonicotinoid compounds are acetamiprid, clothianidin, dinotefuran, imidacloprid, nitenpyram, thiacloprid and thiamethoxam. Preferred neonicotinoid insecticides include clothianidin, thiacloprid, imidacloprid and thiamethoxam. Particularly preferred neonicotinoid insecticides include thiamethoxam, clothianidin and imidacloprid.

Examples of suitable strobilurin compounds are azoxystrobin, pyraclostrobin, picoxystrobin, fluoxastrobin and trifloxystrobin.

The rates of application of the pesticides can vary, for example, according to the specific active ingredient, but is such that the active ingredient(s) provide(s) the desired enhanced action and can be determined by routine experimental trials. Typical application rates, for example, of thiamethoxam can be, for example 50 to 500 g ai/ha, preferably 75 to 400 g ai/ha, more preferably 80 to 350 g ai/ha, especially 100 to 300 g ai/ha.

In an embodiment the stem section has available therefor one or more compounds selected from compounds that exhibit either stimulatory or growth-promoting activity (e.g. nutrients, fertilizers, micronutrient donors, biological agents, inoculants, antibiotics); that exhibit inhibitory activity towards pests and/or pathogens (e.g. a pesticide); and/or exhibit safening activity against pesticide (e.g. a safener); and/or one or more substances that ensures germination and/or storage of the stem section.

The compounds and/or substances can be made available to a stem section by any suitable means. A skilled person would understand that the phrase “made available” or “has available therefor” refers to the stem section being positioned in the proximity of the compounds and/or substances so that the benefits of the compounds and/or substances can be achieved.

Examples of suitable means are treatment of the stem section and encapsulation of the stem section.

Methods for treating compounds, mixtures and compositions thereof on to stem sections include immersing, dressing, coating, pelleting, soaking, tumbling, spraying and injection. The treatment is by a method such that germination of the bud is not induced. Suitably, the treatment method does not cause any damage to the nodes or buds, so that the treatment process does not reduce the ability of buds to form or to emerge into new plants. As a result of the treatment, the active ingredients form part of the stem section, for example, being adhered to the stem section and therefore available for pathogenic and/or pest control, or absorbed into the stem itself through the cut ends. Accordingly, in an embodiment, the present invention provides a pest and/or pathogenic-resistant stem section.

In an embodiment, the stem section is treated before it is sown or planted with one or more compounds selected from compounds that exhibit either stimulatory or growth-promoting activity (e.g. nutrients, fertilizers, micronutrient donors, biological agents, inoculants, antibiotics); compounds that exhibit inhibitory activity towards pests and/or pathogens (e.g. a pesticide); and/or compounds that exhibit safening activity against pesticide (e.g. a safener); so that the sown stem section has been pre-treated with one or more compounds. The treated stem section can, for example, be of any size or dimension provided that the stem section contains at least one bud per stem section. In one embodiment, there is only one bud per stem section.

The stem section can also be treated with one or more substances that ensures germination and/or storage of the stern section.

Suitable substances for pelleting or encapsulating the stem sections include one or more binders, and one or more fibrous materials. Examples of fibrous material are pulp, and fibers from leaf or stem residues, especially of sugar cane such as bagasse. Molasses of the cane sugar may also be used as a sticker (polymer) to bind the fibers for the shaping of the stem sections having one bud.

Binders are useful in the coating and pelleting treatment methods. Binders that are useful in the present invention preferably comprise an adhesive polymer that may be natural or synthetic and is without phytotoxic effect on the buds to be coated. Examples of binders are polyvinyl acetates, polyvinyl acetate copolymers, celluloses, including ethylcelluloses, methylcelluloses, hydromethylcelluloses, hydroxypropylcelluloses and carboxymethylcelluloses; polyvinylpyrolidones, polysaccharides, including starch, modified starch, dextrins, maltodextrins, alginate and chitosans; fats; oils; proteins, including gelatin and zeins; gum arabics; shellacs; vinylidene chloride and vynilidene chloridecopolymers; calcium lignosulfonates; acrylic copolymers; polyvinylacrylates; polyethylene oxide; acrylamide polymers and copolymers; latex; paraffins; polyhydroxyethyl acrylate, methylacrylamide monomers and polychloroprene. Suitably the binder is latex. Suitably treatment of the stem section with a coating or binder serves to retain moisture in the stem section. The coating or binder may be applied to the entire stem section, for example by dipping or immersing the stem in a binder solution, or only to the cut ends of the stem section, for example by painting or spraying.

Pelleting of the stem section helps the bud to withstand mechanical damage during treatment and handling, and also allows easier planting. Suitably the pelleted stem sections are a uniform shape and size to facilitate automated handling and planting. For example, the stem section may be pelleted using a mixture of bagasse and a polymer, leading to an increased shelf life, reduced susceptibility to planting stress, reduced susceptibility to transportation stress, and improved survival of the stem section/bud in dry soil conditions. The pellet may also comprise all fertiliser required at the time of planting.

The coating or binder may be applied to the stem section at the same time as other treatments, such as compounds that exhibit inhibitory activity towards pests and/or pathogens, or compounds that exhibit either stimulatory or growth-promoting activity. In an embodiment, the stem section having one bud is pelleted or coated with one or more compounds and/or one or more substances.

Methods for encapsulating the one or more compounds and/or substances and the stem section can be any suitable technology that maintains the stem section and the compounds and/or substances in proximity. Examples include capsule and “bag” technologies that bio-degrade under specific pre-determined conditions, such as time, moisture or temperature: see for example WO03045139, WO8806839, US620925. The compounds and/or substances are encapsulated with the stem section, optionally the stem section can also be treated with one or more compounds and/or substances.

In a preferred embodiment, the stem section has one bud and one or more compounds selected from compounds that exhibit either stimulatory or growth-promoting activity (e.g., nutrients, fertilizers, micronutrient donors, biological agents, inoculants, antibiotics); compounds that exhibit inhibitory activity towards pests and/or pathogens (e.g. a pesticide); and/or compounds that exhibit safening activity against pesticide (e.g. a safener), and/or one or more substances that ensures germination and/or storage of the stem section are packed in a degradable casing.

In an embodiment, the compound made available to the stem section before planting or sowing can be an insecticide, termiticide, acaricide, miticide, nematicide, molluscicide or fungicide.

Examples of suitable pesticidal compounds are abamectin, cyanoimine, acetamiprid, nitromethylene, nitenpyram, clothianidin, dimethoate, dinotefuran, fipronil, lufenuron, flubendamide, pyripfoxyfen, thiacloprid, fluxofenime, imidacloprid, thiamethoxam, beta cyfluthrin, fenoxycarb, lamda cyhalothrin, diafenthiuron, pymetrozine, diazinon, disulphoton; profenofos, furathiocarb, cyromazin, cypermethrin, tau-fluvalinate, tefluthrin, chlorantraniliprole, flonicamid, metaflumizone, spirotetramat, Bacillus thuringiensis products, azoxystrobin, acibenzolor s-methyl, bitertanol, carboxin, Cu₂O, cymoxanil, cyproconazole, cyprodinil, dichlofluamid, difenoconazole, diniconazole, epoxiconazole, fenpiclonil, fludioxonil, fluoxastrobin, fluquiconazole, flusilazole, flutriafol, furalaxyl, guazatin, hexaconazole, hymexazol, imazalil, imibenconazole, ipconazole, kresoxim-methyl, mancozeb, metalaxyl, R-metalaxyl, mefenoxam, metconazole, myclobutanil, oxadixyl, pefurazoate, paclobutrazole, penconazole, pencycuron, picoxystrobin, prochloraz, propiconazole, pyroquilone, SSF-109, spiroxamin, tebuconazole, thiabendazole, thiram, tolifluamide, triazoxide, triadimefon, triadimenol, trifloxystrobin, triflumizole, triticonazole, uniconazole, a compound of formula A

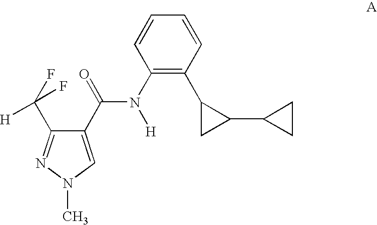

or a tautomer of such a compound, and a compound of formula B

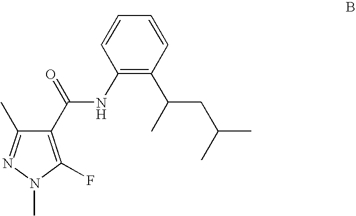

or a tautomer of such a compound.

The compounds of formula A and its manufacturing processes starting from known and commercially available compounds is described in WO 03/074491, WO 2006/015865 and WO 2006/015866. The compound of formula B is described in WO 03/010149 and WO 05/58839.

Mixtures of two or more pesticidal compounds are also envisaged in the present invention for treatment of the stem section.

The sections are treated in an amount sufficient to control the pathogen and/or pest, and can be determined by routine experimental trials.

For example, typical application rates for thiamethoxam may be from 50 to 500 g ai/ha, suitably 75 to 400 g ai/ha, more suitably 80 to 350 g ai/ha, especially 100 to 300 g ai/ha. Typical application rates for abamectin may be from 30 to 300 g ai/ha.

Examples of nutrients include arginine, other amino acid compositions, and inorganic salts such as calcium sulfate CaSO₄, calcium nitrate Ca(NO₃)₂*4H₂O, calcium carbonate CaCO₃, potassium nitrate KNO₃, magnesium sulfate MgSO₄, potassium hydrogen phosphate KH₂PO₄, manganese sulfate MnSO₄, copper sulfate CuSO₄, zinc sulfate ZnSO₄, nickel chloride NiCl₂, cobalt sulfate CoSO₄, potassium hydroxide KOH, sodium chloride NaCl, boric acid H₃BO₃ and metal salts thereof, Na₂MoO₄. Preferred additional nutrients are urea, melamine, potassium oxide, and inorganic nitrates.

Examples of micronutrient donors are Mo, Zn and Co compounds.

Examples of inoculants include nitrogen-fixing agents (such as SBN) and also applicable can be plant inducers (e.g. nodulation factors). For example, the inoculants may include one or more of the following nitrogen fixing bacteria: Gluconacetobacter diazotrophicus, Herbaspirillum seropedicae, Herbaspirillum rubrisubalbicans, Azospirillum amazonense and Burkholderia tropica.

Optionally, the stem sections of the present invention may be treated or coated with biological material such as bacteria, bacterial spores, spores from beneficial fungi, or beneficial nematodes (such as Acetobacter diazotrophicus for its nitrogen fixing properties, Bacillus thuringiensis for its insecticidal toxicity, Rhizobium species, and Verticillium chlamydosporium that has protective effects against root knot nematode).

Examples of biological agents can be selected from one or more of a fungus, bacteria, or other agent, such as NST, which solubilises phosphorus in the soil for improved root uptake.

In an embodiment, the stem section has available therefor one or more substances that ensures germination and/or storage of the stem section.

Suitable substances are those that provide for retention of moisture. Such compounds are known in the art, such as polymers, latex and paraffins. Any binder that seals the stem section, particularly at its cut ends, such that water loss is reduced, is suitable. The binders listed above are suitable for this purpose. In one embodiment, the stem section is treated with a water-retaining gel that absorbs added water and gradually releases it to the stem section over time. Suitable water-retaining gels are routinely used in growing media for pot plants, and are well known to the person skilled in the art.

The means for making available the compounds and/or substances may also have technologies that allow controlled release of the compounds and/or substances.

Controlling, preventing or protecting and its inflections, within the context of the present invention, mean reducing any undesired effect, such as
pathogenic, such as phytopathogenic, especially fungi, infestation or attack of, and
pathogenic damage or pest damage on,
   a plant, part of the plant or stem section to such a level that an improvement is demonstrated.

Each of pesticidal compounds alone or in admixture has very advantageous properties for protecting plants against (i) pathogenic or phytopathogenic fungi attack or infestation, which results in disease and damage to the plant and/or (ii) pest attack or damage; particularly in the instance of plants, the present invention can control or prevent pathogenic damage and/or pest damage on a stem section, parts of plant, plant organs and/or plant grown from the treated stem section. In some cases, control against pest attack or damage also indirectly results in control against pathogenic attack, and vice-a-versa.

In the instance of agriculture, the enhanced actions are found to show an improvement in the growing characteristics of a plant by, for example, higher than expected control of the pathogenic infestation and/or pest damage.

The improvement in the growing (or growth) characteristics of a plant can manifest in a number of different ways, but ultimately it results in a better product of the plant. It can, for example, manifest in improving the yield and/or vigour of the plant or quality of the harvested product from the plant, which improvement may not be connected to the control of diseases and/or pests.

As used herein the phrase “improving the yield” of a plant relates to an increase in the yield of a product of the plant by a measurable amount over the yield of the same product of the plant produced under the same conditions, but without the application of the subject method. It is preferred that the yield be increased by at least about 0.5%, more preferred that the increase be at least about 1%, even more preferred is about 2%, and yet more preferred is about 4%, or more. Yield can be expressed in terms of an amount by weight or volume of a product of the plant on some basis. The basis can be expressed in terms of time, growing area, weight of plants produced, amount of a raw material used, or the like.

As used herein the phrase “improving the vigour” of a plant relates to an increase or improvement of the vigour rating, or the stand (the number of plants per unit of area), or the plant height, or the plant canopy, or the visual appearance (such as greener leaf colour), or the root rating, or percentage emergence, or speed of emergence, or protein content, or number of tillers, or leaf blade size, or less dead basal leaves, or stronger tillers, or less fertilizer needed, or less seeds needed, or more productive tillers, or earlier flowering, or early grain maturity, or less plant verse (lodging), or increased shoot growth, or earlier germination, or any combination of these factors, or any other advantages familiar to a person skilled in the art, by a measurable or noticeable amount over the same factor of the plant produced under the same conditions, but without the application of the subject method.

When it is said that the present method is capable of “improving the yield and/or vigour” of a plant, the present method results in an increase in either the yield, as described above, or the vigour of the plant (or crop), as described above, or both the yield and the vigour of the plant.

Accordingly, the present invention also provides a method of improving the growing characteristics of a crop (or plant) defined in the first aspect, which comprises applying to the stem section defined in the first aspect or locus thereof, a compound as defined in second aspect.

In an embodiment, the present invention provides a method for growing a crop defined in the first aspect having one bud per stem section.

In a particular embodiment, the present invention provides for improved germination of the stem sections having one bud, especially those younger and/or lower stem buds, because of improved root growth.

In an embodiment, one or more neonicotinoid compounds, fipronil, and strobilurin compounds are treated to a stem section having one bud, preferably treated to a bud.

Examples of neonicotinoid compounds are acetamiprid, clothianidin, dinotefuran, imidacloprid, nitenpyram, thiacloprid, thiamethoxam. Preferred neonicotinoid insecticides include clothianidin, thiacloprid, imidacloprid and thiamethoxam. Particularly preferred neonicotinoid insecticides include thiamethoxam, clothianidin and imidacloprid.

Examples of suitable strobilurin compounds are azoxystrobin, pyraclostrobin, picoxystrobin, fluoxastrobin and trifloxystrobin.

The rates of application of the pesticides can vary, for example, according to the specific active ingredient, but is such that the active ingredient(s) provide(s) the desired enhanced action and can be determined by routine experimental trials. For example, typical application rates for thiamethoxam can be from 50 to 500 g ai/ha, suitably from 75 to 400 g ai/ha, more suitably from 80 to 350 g ai/ha, especially from 100 to 300 g ai/ha.

Other pesticides can also be treated to the stem section alone or in combination with one or more neonicotinoid compounds, fipronil, and strobilurin compounds.

Even distribution of the compounds (e.g. active ingredients) and adherence thereof to the stem section is desired during the treatment. Treatment could vary from a thin film (dressing) of the formulation containing the active ingredient(s) on a stem section, where the original size and/or shape are recognizable to an intermediary state (such as a coating) and then to a thicker film (such as pelleting with many layers of different materials (such as carriers, for example, clays; different formulations, such as of other active ingredients; polymers; and colorants) where the original shape and/or size of the seed is no longer recognisable.

The active ingredients are generally applied to the stem section in the form of a conventional formulation.

As with the nature of the formulations, the methods of application, such as drench, spraying, atomizing, dusting, scattering, coating or pouring, are chosen in accordance with the intended objectives and the prevailing circumstances.

The treatment of the defined stem section before planting or sowing would: overcome the disadvantages associated with the need for expensive equipment required for topical in situ application of such compounds to growing plants; reduce the waste associated with the topical in situ application to growing plants; reduce the run-off associated with such topical applications; and reduce the need for repeated re-application of the compounds. This approach also minimizes or eliminates the need for expensive and cumbersome aerial application of products that have sometimes caused environmental concerns. Furthermore, the exposure of the workers to such compounds can be minimized.

Use herein of a term in a singular form also encompasses that term in plural form and vice versa.

The pesticidal compounds described, herein are known and a description of their structure as well as the structures of other pesticides (e.g., fungicides, insecticides, nematicides) can be found in the e-Pesticide Manual, version 3.1, 13th Edition, Ed. CDC Tomlin, British Crop Protection Council, 2004-05.

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections.

### EXAMPLES

### Example 1

### Effect of Formulated ABAMECTIN on “vigour” of Plants Grown from Carretels with Only One Bud and Stalks Having 3 Buds

Greenhouse test with sugarcane buds growing in pots were conducted to study the effect of formulated abamectin regarding “vigour”, tillers, plant height, leaf width and culm diameter on sugar cane Variety RB 86-7515.

### Treatments:

1) carretel with one bud treated with abamectin
2) carretel with one bud treated with abamectin+15% v/v of polymer
3) stalk with 3 buds untreated
4) carretel with one bud untreated
5) carretel with one bud untreated+15% v/v of polymer
6) stalk with 3 buds treated with abamectin.

Twenty 1 L pots were filled with 16 L of clay soil, 1.6 L of sand and 8 g of nitrogen, 28 g of phosphorus (P₂O₅) and 16 g of potassium (K₂O). Six carretels of 4 cm length with only one bud were planted in each pot (treatments 1, 2, 4 and 5). Two stalks with three buds were planted in each control pot (treatments 3 and 6) to simulate conventional planting. A layer of 3 cm of soil was used to cover the carretels. The carretels of treatments 1, 2 were treated with 200 ml/ha of abamectin, by dipping the carretels in the slurry for 3 min, based on the uptake capacity of carretels. The stalks were treated simulating a furrow application at 100 L.ha⁻¹ of slurry. Experimental design adopted was randomized complete block with four replications. The pots were maintained with adequate moisture during the test. Evaluations of: plant height, leaf width, culm diameter and number of tillers were done at 20, 30 and 42 days after planting. The results are presented in Table 1.

TABLE 1
Evaluation of tillers, plant height, leaf width and culm diameter of the
treatments with abamectin in sugarcane carretels with one bud in direct
comparison with stalks of 3 buds. Evaluations at 42 days after planting.

| | | | | | Leaf | Culm |
|---|---|---|---|---|---|---|
| | Sugarcane | Rate | Tillers | Plant Height | Width | Diameter |
| Treatments | “Seed” | (g ai/ha) | (number) | (cm) | (mm) | (mm) |
| ABAMECTIN | Carretel with | 100 | 14 | 22.22 | 14.58 | 7.67 |
| | one bud | | | | | |
| ABAMECTIN + | Carretel with | 100 | 12 | 26.15 | 15.08 | 8.0 |
| Polymer | one bud | | | | | |
| ABAMECTIN | Stalk with 3 | 100 | 19.5 | 22.54 | 20.33 | 12.25 |
| | buds | | | | | |
| Untreated + | Carretel with | 0.0 | 12 | 26.02 | 16.16 | 8.65 |
| Polymer | one bud | | | | | |
| Untreated | Carretel with | 0.0 | 11.5 | 26.23 | 14.49 | 7.92 |
| | one bud | | | | | |
| Untreated | Stalk with 3 | 0.0 | 12 | 25.98 | 20.33 | 11.25 |
| | buds | | | | | |

The results show that the agrochemical formulation resulted in an increase in the number of tillers in comparison with the control but a decrease in plant height. The addition of polymer reversed these effects, at least to some extent.

### Example 2

### Effect of a Formulated BISAMIDE INSECTICIDE on “Vigour” of Plants Grown from Carretels with Only One Bud and Stalks Having 3 Buds

Greenhouse tests with sugarcane buds growing in pots were conducted to study the effect of a formulated bisamide insecticide (the formulation) on vigour, evaluating tillers, plant height, leaf width and culm diameter on sugar cane Variety RB 86-7515.

### Treatments:

1) carretel with one bud treated with the formulation
2) carretel with one bud treated with the formulation+15% v/v of polymer
3) stalk with 3 buds untreated
4) carretel with one bud untreated
5) carretel with one bud untreated+15% v/v of polymer
6) stalk with 3 buds treated with the formulation.

Twenty 1 L pots were filled with 16 L of clay soil, 1.6 L of sand and 8 g of nitrogen, 28 g of phosphorus (P₂O₅) and 16 g of potassium (K₂O). Six carretels of 4 cm length with one bud were planted in each pot (treatments 1, 2, 4 and 5). Two stalks with three buds were planted in the control pots (treatments 3 and 6) to simulate conventional planting. A layer of 3 cm of soil was used to cover the carretels. The carretels of treatments 1 and 2 were treated with the formulation, by dipping the carretels in the slurry for 3 min, based on the uptake capacity of carretels. The stalks were treated simulating a furrow application at 100 L.ha⁻¹ of slurry. Experimental design adopted was randomized complete block with four replications. The pots were maintained with adequate moisture during the test. Evaluations of: plant height, leaf width, culm diameter and number of tillers were done at 20, 30 and 42 days after planting. The results are presented in Table 2.

TABLE 2
Evaluation of number of tillers, plant height, leaf width and culm diameter of
the treatments with the formulation in sugarcane carretels with one bud in direct
comparison with 3 buds stalks. Evaluations at 42 days after planting.

| | | | | | Leaf | Culm |
|---|---|---|---|---|---|---|
| | Sugarcane | Rate | Tillers | Plant Height | Width | Diameter |
| Treatments | “Seed” | (g ai/ha) | (number) | (cm) | (mm) | (mm) |
| Formulation | Carretel with | 150 | 16.25 | 22.49 | 15.27 | 7.33 |
| | one bud | | | | | |
| Formulation + | Carretel with | 150 | 13.75 | 25.0 | 14.58 | 7.75 |
| polymer | one bud | | | | | |
| Formulation | Stalk with 3 | 150 | 14.75 | 23.01 | 20.4 | 10.67 |
| | buds | | | | | |
| Untreated + | Carretel with | 0.0 | 12.75 | 25.61 | 15.0 | 8.0 |
| polymer | one bud | | | | | |
| Untreated | Carretel with | 0.0 | 12.5 | 25.1 | 14.16 | 7.66 |
| | one bud | | | | | |
| Untreated | Stalk with 3 | 0.0 | 11.5 | 27.78 | 19.91 | 10.82 |
| | buds | | | | | |

The formulated bisamide insecticide increased the number of tillers of the plant in comparison with untreated controls, whether for the 1 bud or 3 bud systems, and likewise decreased plant height. The addition of polymer reduced these effects, at least to some extent.

### Example 3

### Effect of Formulated THIAMETHOXAM on “Vigour” of Plants Grown from Carretels with Only One Bud and Stalks Having 3 Buds

Greenhouse test with sugarcane buds growing in pots were conducted to study the performance of a formulated agrochemical formula comprising thiamethoxam (35%) on “vigour”, comprising an evaluation on tillers, plant height, leaf width and culm diameter. This trial was conducted on Variety RB 86-7515.

### Treatments:

1) carretel with one bud treated with the equivalent of 1200 ml. ha⁻¹ of a 35% w/w solution of thiamethoxam (the formulation)
2) carretel with one bud treated with the equivalent of 1200 ml. ha⁻¹ of a 35% w/w solution of thiamethoxam+15% v/v of polymer
3) stalk with 3 buds untreated
4) carretel with one bud untreated
5) carretel with one bud untreated+15% v/v of polymer
6) stalk with 3 buds treated with the equivalent of 1200 ml. ha⁻¹ of a 35% w/w solution of thiamethoxam.

Twenty 1 L pots were filled with 16 L of clay soil, 1.6 L of sand and 8 g of nitrogen, 28 g of phosphorus (P₂O₅) and 16 g of potassium (K₂O). Six carretels of 4 cm length with one bud were planted in each pot (treatments 1, 2, 4 and 5). Two stalks with three buds were planted in each control pot (treatments 3 and 6) to simulate conventional planting. A layer of 3 cm of soil was used to cover the carretels. The carretels of treatments 1 and 2 were treated with 420 g ai.ha⁻¹ of thiamethoxam, by dipping the carretels in the slurry for 3 min. The stalks were treated simulating a furrow application at 100 L.ha⁻¹ of slurry. Experimental design adopted was randomized complete block with four replications. The pots were maintained with adequate moisture during the test. Evaluations of: plant height, leaf width, culm diameter and number of tillers were done at 20, 30 and 42 days after planting. The results are presented in Table 3.

TABLE 3
Evaluation of tillers, plant height, leaf width and culm diameter of the
treatments with thiamethoxam in sugarcane carretels with one bud in
direct comparison with 3 bud stalk. Evaluations at 42 days after planting.

| | | | | | Leaf | Culm |
|---|---|---|---|---|---|---|
| | Sugarcane | Rate | Tillers | Plant Height | width | Diameter |
| Treatments | “Seed” | (g ai/ha) | (number) | (cm) | (mm) | (mm) |
| Thiamethoxam | Carretel with | 420 | 15.5 | 23.67 | 14.07 | 7.97 |
| | one bud | | | | | |
| Thiamethoxam + | Carretel with | 420 | 13.0 | 26.42 | 15.1 | 8.5 |
| Polymer | one bud | | | | | |
| Thiamethoxam | Stalk with 3 | 420 | 14.75 | 26.52 | 19.5 | 10.17 |
| | buds | | | | | |
| Untreated + | Carretel with | 0.0 | 12.25 | 27.25 | 15.57 | 9.12 |
| Polymer | one bud | | | | | |
| Untreated | Carretel with | 0.0 | 12.75 | 26.28 | 12.32 | 7.6 |
| | one bud | | | | | |
| Untreated | Stalk with 3 | 0.0 | 10.75 | 28.71 | 19.65 | 10.0 |
| | buds | | | | | |

Carretels treated with thiamethoxam per se exhibited a reduced plant height but increased number of tillers in comparison with the corresponding controls. The addition of polymer reversed this effect, at least to some extent.

### Example 4

### Effect of Formulated CLOTHIANIDIN on “Vigour” of Plants Grown from Carretels with Only One Bud and Stalks Having 3 Buds

Greenhouse test with sugarcane buds growing in pots were conducted to study the effect of clothianidin equivalent to an application rate of 200 g ai. ha⁻¹ on “vigour”, evaluating tillers, plant height, leaf width and culm diameter. This trial was conducted on sugar cane Variety RB 86-7515.

### Treatments:

1) carretel with one bud treated with clothianidin (200 g ai. ha⁻¹)
2) carretel with one bud treated with clothianidin (200 g ai. ha⁻¹)+15% v/v of polymer
3) stalk with 3 buds untreated
4) carretel with one bud untreated+15% v/v of polymer
5) carretel with one bud untreated
6) stalk with 3 buds treated with clothianidin (200 g ai. ha⁻¹).

Twenty 1 L pots were filled with 16 L of clay soil, 1.6 L of sand and 8 g of nitrogen, 28 g of phosphorus (P₂O₅) and 16 g of potassium (K₂O). Six carretels of 4 cm length with one bud were planted in each pot (treatments 1, 2, 4 and 5). Two stalks with three buds were planted in each control pot (treatments 3 and 6) to simulate conventional planting. A layer of 3 cm of soil was used to cover the carretels. The carretels of treatments 1 and 2 were treated with 200 g ai.ha⁻¹ of clothianidin, by dipping the carretels in a slurry for 3 min. The stalks were treated simulating a furrow application at 100 L.ha⁻¹ of slurry. The pots were maintained with adequate moisture during the test. Evaluations of: plant height, leaf width, culm diameter and number of tillers were performed at 20, 30 and 42 days after planting. The results are presented in Table 4.

TABLE 4
Evaluation of tillers, plant height, leaf width and culm diameter of the
treatments with clothianidin in sugarcane carretels with one bud in
direct comparison with 3 buds stalk. Evaluations at 42 days after planting.

| | | | | | Leaf | Culm |
|---|---|---|---|---|---|---|
| | Sugarcane | Rate | Tillers | Plant Height | Width | Diameter |
| Treatments | “Seed” | (g ai/ha) | (number) | (cm) | (mm) | (mm) |
| Clothianidin | Carretel with | 200 | 14.5 | 25.02 | 17.17 | 8.75 |
| | one bud | | | | | |
| Clothianidin + | Carretel with | 200 | 11.75 | 28.66 | 17.5 | 8.83 |
| polymer | one bud | | | | | |
| Clothianidin | Stalk with 3 | 200 | 17.0 | 22.73 | 21.5 | 11.25 |
| | buds | | | | | |
| Untreated + | Carretel with | 0.0 | 10.5 | 27.1 | 15.17 | 6.91 |
| polymer | one bud | | | | | |
| Untreated | Carretel with | 0.0 | 11.75 | 26.86 | 15.35 | 7.0 |
| | one bud | | | | | |
| Untreated | Stalk with 3 | 0.0 | 9.25 | 25.54 | 20.25 | 10.25 |
| | buds | | | | | |

Carretels treated with clothianidin per se had an increased number of tillers in comparison with the corresponding control experiment (untreated, one-bud) but a slight reduction in the plant height; polymer treatment reversed the effects of the formulated active material, at least to some extent.

### Example 5

### Effect of Formulated FIPRONIL on “Vigour” of Plants Grown from Carretels with Only One Bud and Stalks Having 3 Buds

Greenhouse test with sugarcane buds growing in pots were conducted to study the effect of fipronil equivalent to an application rate of 200 g. ha⁻¹ on “vigour”, evaluating tillers, plant height, leaf width and culm diameter on sugar cane Variety RB 86-7515.

### Treatments:

1) carretel with one bud treated with fipronil (200 g. ha⁻¹)
2) carretel with one bud treated with fipronil (200 g. ha⁻¹)+15% v/v of polymer
3) stalk with 3 buds untreated
4) carretel with one bud untreated
5) carretel with one bud untreated+15% v/v of polymer
6) stalk with 3 buds treated with fipronil (200 g. ha⁻¹).

Twenty 1 L pots were filled with 16 L of clay soil, 1.6 L of sand and 8 g of nitrogen, 28 g of phosphorus (P₂O₅) and 16 g of potassium (K₂O). Six carretels of 4 cm length with one bud were planted in each pot (treatments 1, 2, 4 and 5). Two stalks with three buds were planted in each control pot (treatments 3 and 6) to simulate conventional planting. A layer of 3 cm of soil was used to cover the carretels. The carretels of treatments 1 and 2 were treated with 200 g ai.ha⁻¹ of fipronil, by dipping the carretels in the slurry for 3 min, based on the uptake capacity of carretels. The stalks were treated simulating a furrow application at 100 L.ha⁻¹ of slurry. Experimental design adopted was randomized complete block with four replications. The pots were maintained with adequate moisture during the test. Evaluations of: plant height, leaf width, culm diameter and number of tillers were done at 20, 30 and 42 days after planting. The results are presented in Table 5.

TABLE 5
Evaluation of tillers, plant height, leaf width and culm diameter of the
treatments with fipronil in sugarcane carretels with one bud in direct
comparison with 3 bud stalk. Evaluations at 42 days after planting.

| | | | | Plant | Leaf | Culm |
|---|---|---|---|---|---|---|
| | Sugarcane | | Tillers | Height | Width | Diameter |
| Treatments | “Seed” | Rate (g ai/ha) | (number) | (cm) | (mm) | (mm) |
| Fipronil | Carretel with | 200 | 17.25 | 20.62 | 14.16 | 8.69 |
| | one bud | | | | | |
| Fipronil + | Carretel with | 200 | 16.5 | 26 | 14.49 | 8.75 |
| Polymer | one bud | | | | | |
| Fipronil | Stalk with 3 | 200 | 16.5 | 22.12 | 18.91 | 10.75 |
| | buds | | | | | |
| Untreated + | Carretel with | 0.0 | 12.75 | 24.29 | 13.99 | 8.33 |
| Polymer | one bud | | | | | |
| Untreated | Carretel with | 0.0 | 11.25 | 25.44 | 13.41 | 8.16 |
| | one bud | | | | | |
| Untreated | Stalk with 3 | 0.0 | 10 | 24.57 | 21.91 | 10.53 |
| | buds | | | | | |

The treatment of the sugar cane material with fipronil decreased the height of the resultant plants in comparison with the control, but increased the number of tillers present on the plants. These effects were reversed, at least to some extent, by the presence of the polymer.

### Example 6

### Effect of Formulated IMIDACLOPRID on “Vigour” of Plants Grown from Carretels with Only One Bud and Stalks Having 3 Buds

Greenhouse tests with sugarcane buds growing in pots were conducted to study the performance of formulated Imidacloprid at the equivalent to the commercial application rate 1200 g. ha⁻¹ regarding “vigour”, evaluating tillers, plant height, leaf width and culm diameter on sugar cane Variety RB 86 7515.

### Treatments:

1) carretel with one bud treated with Imidacloprid (1200 g. ha⁻¹)
2) carretel with one bud treated with Imidacloprid (1200 g. ha⁻¹)+15% v/v of polymer
3) stalk with 3 buds untreated
4) carretel with one bud untreated
5) carretel with one bud untreated+15% v/v of polymer
6) stalk with 3 buds treated with Imidacloprid (1200 g. ha⁻¹).

Twenty 1 L pots were filled with 16 L of clay soil, 1.6 L of sand and 8 g of nitrogen, 28 g of phosphorus (P₂O₅) and 16 g of potassium (K₂O). Six carretels of 4 cm length with one bud were planted in each pot (treatments 1, 2, 4 and 5). Two stalks with three buds were planted in each control pot (treatments 3 and 6) to simulate conventional planting. A layer of 3 cm of soil was used to cover the carretels. The carretels of treatments 1 and 2 were treated with 1200 g ai.ha⁻¹ of imidacloprid, by dipping the carretels in the slurry for 3 min, based on the uptake capacity of carretels. The stalks were treated simulating a furrow application at 100 L.ha⁻¹ of slurry. Experimental design adopted was randomized complete block with four replications. The pots were maintained with adequate moisture during the test. Evaluations of: plant height, leaf width, culm diameter and number of tillers were done at 20, 30 and 42 days after planting. The results are presented in Table 6.

TABLE 6
Evaluation of tillers, plant height, leaf width and culm diameter of the
treatments with imidacloprid in sugarcane carretels with one bud in
direct comparison with 3 bud stalk. Evaluations at 42 days after planting.

| | | | | | Leaf | Culm |
|---|---|---|---|---|---|---|
| | Sugarcane | Rate | Tillers | Plant Height | Width | Diameter |
| Treatments | “Seed” | (g ai/ha) | (number) | (cm) | (mm) | (mm) |
| Imidacloprid | Carretel with | 1200 | 15.25 | 24.0 | 15.91 | 8.75 |
| | one bud | | | | | |
| Imidacloprid + | Carretel with | 1200 | 13.0 | 26.85 | 16.16 | 7.66 |
| Polymer | one bud | | | | | |
| Imidacloprid | Stalk with 3 | 1200 | 12.0 | 23.49 | 20.16 | 9.49 |
| | buds | | | | | |
| Untreated + | Carretel with | 0.0 | 12.5 | 26.62 | 14.25 | 8.32 |
| Polymer | one bud | | | | | |
| Untreated | Carretel with | 0.0 | 11.5 | 23.71 | 13.58 | 7.5 |
| | one bud | | | | | |
| Untreated | Stalk with 3 | 0.0 | 12.0 | 25.07 | 19.0 | 8.75 |
| | buds | | | | | |

The effect of the formulated imidacloprid was less pronounced than with the other active ingredients. Nevertheless it resulted in an increase in the number of tillers in comparison with the control, an effect which once again was decreased in the polymer treated situation.

### Example 7

### Effect of Formulated AZOXYSTROBIN+CYPROCONAZOL on “Vigour” of Plants Grown from Carretels with Only One Bud and Stalks Having 3 Buds

Greenhouse test with sugarcane buds growing in pots were conducted to study the performance of a formulation comprising Azoxystrobin and cyproconazol (azoxystrobin 20%+cyproconazol 8%) (hereinafter “formulation”) on vigour, evaluating tillers, plant height, leaf width and culm diameter of sugar cane Variety RB 86-7515.

### Treatments:

1) carretel with one bud treated with the formulation (300 ml. ha⁻¹)
2) carretel with one bud treated with the formulation (300 ml. ha⁻¹)+15% v/v of polymer
3) stalk with 3 buds untreated,
4) carretel with one bud untreated
5) carretel with one bud untreated+15% v/v of polymer
6) stalk with 3 buds treated with the formulation (300 ml. ha⁻¹).

Twenty 1 L pots were filled with 16 L of clay soil, 1.6 L of sand and 8 g of nitrogen, 28 g of phosphorus (P₂O₅) and 16 g of potassium (K₂O). Six carretels of 4 cm length with one bud were planted in each pot (treatments 1, 2, 4 and 5). Two stalks with three buds were planted in each referred pot (treatments 3 and 6) to simulate conventional planting. A layer of 3 cm of soil was used to cover the carretels. The carretels of treatments 1 and 2 were treated with 84 g ai.ha⁻¹ of axoxystrobin+cyproconazol by dipping the carretels in the slurry for 3 min, based on the uptake capacity of carretels. The stalks were treated simulating a furrow application at 100 L.ha⁻¹ of slurry. Experimental design adopted was randomized complete block with four replications. The pots were maintained with adequate moisture during the test. Evaluations of: plant height, leaf width, culm diameter and number of tillers were done at 20, 30 and 42 days after planting. The results are presented in Table 7.

TABLE 7
Evaluation of tillers, plant height, leaf width and culm diameter of the treatments
with azoxystrobin + cyproconazol in sugarcane carretels with one bud in
direct comparison with 3 bud stalk. Evaluations at 42 days after planting.

| | | | | | Leaf | Culm |
|---|---|---|---|---|---|---|
| | Sugarcane | Rate | Tillers | Plant Height | Width | Diameter |
| Treatments | “Seed” | (g ai/ha) | (number) | (cm) | (mm) | (mm) |
| Formulation | Carretel with | 84 | 12.75 | 20.84 | 15.15 | 8.02 |
| | one bud | | | | | |
| Formulation + | Carretel with | 84 | 13.5 | 23.8 | 16.12 | 8.67 |
| Polymer | one bud | | | | | |
| Formulation | Stalk with 3 | 84 | 15.25 | 28.36 | 18.82 | 11.25 |
| | buds | | | | | |
| Untreated + | Carretel with | 0.0 | 10.0 | 26.64 | 15.2 | 8.82 |
| Polymer | one bud | | | | | |
| Untreated | Carretel with | 0.0 | 10.5 | 26.55 | 14.8 | 7.75 |
| | one bud | | | | | |
| Untreated | Stalk with 3 | 0.0 | 12.25 | 24.48 | 19.87 | 11.5 |
| | buds | | | | | |

Treatment of the sugar cane material with the formulation increased the number of tillers but reduced the height of the plants in comparison with the control plants—at least for the one bud only system. The addition of polymer reduced both the increase in tiller number and decreased the plant height brought about by the formulation treatment.

### Example 8

### Effect of Formulated ABAMECTIN and THIAMETHOXAM on “Vigour” of Plants Grown from Carretels with Only One Bud and Stalks Having 3 Buds

A field test with sugarcane buds was conducted to study the performance of a formulation comprising thiamethoxam (35%) and a formulation comprising abamectin (50%) for termite (Heterothermes sp) control. This insect species feeds on the sugar cane reserve tissues and causes a drastic reduction in the emergence and development of shoots and roots. In addition the treatments included an application of a mixture of fungicides (azoxystrobin (20% v/v) and cyproconazole (8% v/v)).

The sugarcane carretels of 4 cm and only one bud, Variety BR 86-7515, were treated by dipping in a slurry of agrochemicals for 3 minutes. Where indicated, 15% v/v latex was added to the slurry. The test area was chosen previously to be sure that it was with very high termite infestation. Plots measured 4 rows of sugarcane by 5 m length. Row space was 1.5 m. Each row received 8 carretels per meter, totalling 160 carretels per plot. Controls of untreated carretels and stalk (3 buds) were included, as standard was selected stalk with 40 cm treated in furrow with fipronil, carbofuran and triadimenol. Experimental design of randomized complete block (RCB) was adopted with 4 replications. Evaluations of % emergence of sugarcane were done only in two central rows at 21 and 42 days after application. The results are presented in Table 8.

TABLE 8
Evaluation of % emergence of sugarcane carretels with one bud treated with
abamectin + thiamethoxam for controlling termites (Heterothermes sp).

| | Sugarcane | Rate | % Emergence | % Emergence |
|---|---|---|---|---|
| Treatments | “Seed” | (g ai/ha) | 21 DAA | 42 DAA |
| Untreated | Carretel with | 0 | 10.6 | 21.0 |
| | one bud | | | |
| Thiamethoxam + | Carretel with | 70 + 100 | 53.1 | 64.1 |
| abamectin + latex | one bud | | | |
| Thiamethoxam + | Carretel with | 140 + 100 | 55.6 | 69.8 |
| abamectin + latex | one bud | | | |
| Thiamethoxam + | Carretel with | 210 + 100 | 66.3 | 71.6 |
| abamectin + latex | one bud | | | |
| Thiamethoxam + | Carretel with | 280 + 100 | 54.8 | 62.9 |
| abamectin + latex | one bud | | | |
| Thiamethoxam + | Carretel with | 350 + 0100 | 55.4 | 67.3 |
| abamectin + latex | one bud | | | |
| Thiamethoxam + | Carretel with | 420 + 100 | 53.8 | 63.8 |
| abamectin + latex | one bud | | | |
| Fipronil + carbofuran + | Stalk with 3 | 200 + 2100 + | 24.5 | 47.4 |
| triadimenol + latex | buds | 150 | | |
| Untreated | Stalk with 3 | 0 | 15.6 | 37.3 |
| | buds | | | |

Emergence was evaluated initially 21 days after planting: the concoction of active ingredients in the formulation provided for a very good rate—much better than the control (untreated material), and better than a present standard treatment (comprising the application of fipronil, carbofuran and triadimenol). In addition an assessment of damage to the plants caused by the insect pest showed that the formulation tested provided a degree of control similar to that of the standard. Percentage emergence means the percentage of the carretels that emerged in relation to the number planted.

### Example 9

### Effect of POLYMER on “Vigour” of Plants Grown from Carretels with Only One Bud and Stalks Having 3 Buds

Water losses (dehydration) reduces the emergence of single bud carretels: polymers may be able to reduce the dehydration thereby improving emergence levels in the field. To prove this concept a field test with sugarcane carretels with one bud was conducted to demonstrate the importance of polymer in the slurry treatment of one single bud in sugarcane.

### Material and Methods

The field trials were conducted under excellent environmental conditions. Sugarcane carretels of 4 cm and only one bud, variety BR 86-7515, were treated by dipping the carretels in a slurry (chemicals compounds±polymer, the polymer used was a emulsion of acrylic resin at 7% of concentration), for 3 minutes. Plot size: 4 rows of sugarcane by 5 m long; row space: 1.5 m; planting 8 carretels/m. One check of untreated carretels was included. Experimental design of randomized complete block (RCB) was adopted with 4 replications. Evaluations of % emergence of sugarcane carretels were done in two central rows at 21 and 35 days after application.

If an assessment of emergence is made after only 21 days there is little difference between the presence and absence of polymer, except in the case that the sugar cane material has also been treated with very high concentration of formulated agrochemicals, in which case the polymer appears to substantially increase the emergence frequency. At 35 days after planting, however, the percentage emergence in the presence of polymer is greater than that in its absence, with once again a “possible safening” effect being observed if the sugar cane had been treated with relatively high levels of formulated agrochemicals.

### SUMMARY OF EXAMPLES

As a generality treatment of sugar cane stalks having 3 buds, and in particular carretels having only one bud, with formulated agrochemicals (such as fungicides, insecticides and/or nematicides) increased the number of tillers on the resulting plant, but tended to reduce the plant height. Addition of polymer, with the aim of reducing fluid and/or nutrient loss from the carretels, generally reversed the effects seen by the treatment of the formulated active ingredients although in most cases the number of tillers remained higher than control levels.

An assessment of “vigour” involves a consideration of a number of parameters, an increase or decrease in any one of which is perhaps insufficient to provide a designation of an improvement, or otherwise, per se. What is important in making an assessment is the net effect of the treatment on the various parameters combined—something which is not necessarily so obvious from a statistical analysis of the data obtained in respect of each of them. The man skilled in the art knows an improvement when he sees it—be it in the glasshouse or field—and this improvement is not necessarily so readily apparent from lists of data provided in respect of the various parameters assessed.

Accordingly, although the data for the thiamethoxam trials appears to be, in many respects and as a generality, analogous to that obtained in respect of other formulated agrochemicals—as a practical matter the thiamethoxam treatment is preferred because it provided an increase in “quality” greater than the other treatments (in so far as they can be compared with each other, having regard to their mode of action, application rate and presence in the compositions containing them of standard formation components) not apparent from the measurements made in respect of the listed parameters.

Further embodiments of the invention now disclosed will be obvious to the skilled man. For example, where the stem sections are to be coated with plant growth promoting compounds or pest control compounds, an immersion technique may preferably be employed compared with other, known coating techniques. It may be desirable to store the stem sections prior to planting, in which case dry and cool storage conditions are preferred in comparison with humid warm ones, the precise conditions depending on the crop and time it is intended to store the material prior to planting.

1. A method of growing a gramineous crop plant comprising the steps of
a) providing a stem section of a gramineous crop plant which section comprises at least one node,
b) planting said section, and
c) growing a gramineous crop plant from said planted stem section,
wherein the stem section is stored at a temperature below 15° C. before it is planted.

2. A method of propagating a gramineous crop plant comprising the steps of
a) providing more than one stem section from a gramineous crop plant by cutting the stem of said plant, wherein each section comprises at least one node,
b) planting said multiple sections, and
c) growing gramineous crop plants from said planted stem sections,
wherein the stem sections are stored at a temperature below 15° C. before it is planted.

3. A method according to claim 1, wherein the stem section comprises only one node.

4. A method according to claim 1, wherein the stem section is from about 2 to about 12 cm in length.

5. A method according to claim 4, wherein the stem section is from about 3 to about 8 cm in length.

6. A method according to claim 1, wherein the stem section is planted in an essentially horizontal position in a furrow.

7. A method according to claim 1, wherein the stem section is stored at a temperature below 10° C. before it is planted.

8. A method according to claim 7, wherein the stem section is stored at a temperature from about 6 to about 8° C. before it is planted.

9. A method of growing a gramineous crop plant comprising the steps of
a) preparing a stem section of a gramineous crop plant by cutting the stem of said plant, wherein the section comprises only one node,
b) planting said section, and
c) growing a gramineous crop plant from said planted stem section,
wherein the stem section is planted within about 2 months after it has been prepared.

10. A method of propagating a gramineous crop plant comprising the steps of
a) preparing more than one stem section from a gramineous crop plant by cutting the stem of said plant, wherein each section comprises only one node,
b) planting said multiple sections, and
c) growing gramineous crop plants from said planted stem sections,
wherein the stem sections are planted within about 2 months after they have been prepared.

11. A method according to claim 9, wherein the stem section is from about 2 to about 12 cm in length.

12. A method according to claim 11, wherein the stem section is from about 3 to about 8 cm in length.

13. A method according to claim 9, wherein the stem section is planted in an essentially horizontal position in a furrow.

14. A method according to claim 9, wherein the stem section is planted within about 1 month after it has been prepared.

15. A method according to claim 14, wherein the stem section is planted within about 2 weeks after it has been prepared.

16. A method according to claim 1, wherein the gramineous crop plant is selected from the group consisting of Saccharum spp., Sorghum spp., and bamboo.

17. A method according to claim 16, wherein the gramineous crop plant is Saccharum spp.

18. A stem section of a gramineous crop plant, characterized in that it
a) comprises at least one node, and
b) has been stored at a temperature below 15° C. before it is planted.

19. A stem section according to claim 18 that comprises only one node.

20. A stem section according to claim 19, wherein the stem section is stored at a temperature below 10° C. before it is planted.

21. A stem section according to claim 20, wherein the stem section is stored at a temperature from about 6 to about 8° C. before it is planted.

22. A stem section of a gramineous crop plant that has been prepared by cutting the stem of said plant, characterized in that it
a) comprises only one node, and
b) is planted within about 2 months after it has been prepared.

23. A stem section according to claim 22, wherein the stem section is planted within about 1 month after it has been prepared.

24. A stem section according to claim 23, wherein the stem section is planted within about 2 weeks after it has been prepared.

25. A stem section according to claim 18 that is from about 2 to about 12 cm in length.

26. A stem section according to claim 25 that is from about 3 to about 8 cm in length.

27. A stem section according to claim 18, wherein the gramineous crop plant is selected from the group consisting of Saccharum spp., Sorghum spp., and bamboo.

28. A stem section according to claim 27, wherein the gramineous crop plant is Saccharum spp.

29. (canceled)
